(19) United States
(12) Patent Application Publication
(10) Pub. No.: US 2002/0098532 A1
(43) Pub. Date: Jul. 25, 2002

(54) One step test for abortion safety based on detection of 2 IU/ml or more of gonadtropin in a urine sample

(76) Inventor: Hsian Chiang Yee, Seattle, WA (US)

Correspondence Address: Robert W. Jenny, 77 Cascade Key, Bellevue, WA, 98006, US

(21) Appl. No.: 09/727,149

(22) Filed: Dec. 1, 2000

(51) Int. Cl. G01N 33/53; G01N 33/537; G01N 33/543

(52) U.S. Cl. 435/7.92

(57) ABSTRACT

The subject invention is an immunoassay for the qualitative determination of human chrionic gonadtroysin (hCG) in urine as an aid in the diagnosis of a certain stage of pregnancy. It is intended for professional and laboratory use. The test device is a dipstrip having a dipping end. Results are indicated by coloration of two bands across a clear area of the dipstrip, one band being coated with a reagent such as hCG antigens and the other with a reagent such as goat/rabbit polyclonal antibodies. The urine sample migrates through the absorbent material in the dipstrip, past an antibody gold conjugate and the two bands. The bands are not visible until a test is in progress and the coloration and/or lack of coloration of one or both bands indicates the test results.

### BACKGROND OF THE INVENTION

1. Field

The subject invention is in the field of methods, tests and techniques for determining whether or not a human has a particular disease, condition, illness or affliction, determining the severity of these and determining the degree of certainty of the determinations. In particular, the subject invention is in the field of methods, tests and techniques for determining an optimal time period during which a woman may have an abortion or the abortion is completed.

2. Prior Art

Pregnancy testing in the 1920s and 1930s involved using a woman's urine and one or more of various animals such as rats, toads and rabbits. These tests could confirm pregnancy approximately two months after a missed period. Between 1940 and 1960 methods called bioassays used a hemagglutination and latex agglutination and were simpler than prior testing and provided earlier results. However, analytic sensitivity was limited to 150-1000 mIU/ml. In about 1960 bioassays were replaced by immunoassays. When a woman becomes pregnant her body produces a hormone called hCG (human Chorionic Gonadotropin) which appears in her urine. Production of this hormone increases throughout the first trimester of pregnancy. Sensitive radioimmunoassay, immunoradiometric and enzyme immunoassay become available and allow an accurate and precise quantification of hCG by use of highly specific antibodies. These assays (tests) detect pregnancy by the first day of a missed menstrual period. Monoclonal antibodies to hCG have been developed and improve the specificity of these three assays by providing a supply of the highly specific antibodies needed.

Many pregnancy tests are now commercially available and provide rapid, sensitive determination of pregnancy in early stages. However, there is no lateral flow test kit for rapid tests of this kind, i.e. hcg 2 IU/ml, and none is especially adapted to detecting pregnancy during the time period in which abortion is safest, the adaptation being the capability to detect 2 IU/ml or less of hCG in urine. Abortion is considered safe if no more than 2 IU/ml of hCG in urine is detected. At that stage the baby is not yet well developed. The hcG IU/ml also detects whether or not the abortion is completed. The only currently available test with that capability is the Wampole immunochemical latex agglutination slide test with a cut-off level of 2 IU/ml and a variation in sensitivity of at least twofold dilution can be expected. Latex agglutination is slow and the accuracy level is undesirable. Accordingly, the objective of the subject invention is to provide a rapid test for determining an optimal time period during which a woman may have an abortion or if the abortion is completed.

### SUMMARY OF THE INVENTION

The subject invention is a test which enables rapid detection of 2 IU/ml or more of human chorionic gonadtropin (hCG) in a urine sample. The test is based on antibody function. The test kits for using the test each comprise a dipstrip and, in another embodiment, a cassette and a dropper. The dipstrip comprises a backing sub-strip, an absorbent materials sub-strip, a cover sub-strip having a clear portion termed a window and a portion of labeled antibody gold conjugate. The sub-strips are laminated with a short portion of the absorbent materials exposed at the dipping end of the dipstrip. The window may have a T and a C on its inner side indicating the locations of the test band and control band respectively.

In performing a test the dipping end of the dipstrip is dipped into a urine sample. The portion of labeled antibody gold conjugate is held between the absorbent materials sub-strip and the cover sub-strip and between the window and the dipping end. The dipstrip may be installed in a cassette having an opening which corresponds to the window of the installed dipstrip. There may be a T and a C on the outside of the cassette, possibly correlating with the T and C on the window. When the dipstrip is installed in a cassette a test is done by applying the sample, by a dropper, into a well opening which corresponds to the dipping end of the dipstrip. The test bands are not visible except under certain test conditions and are transverse to the strip.

The test band is coated with hCG antigen. The control band is coated with reagents such as goat/rabbit polyclonal antibodies against monoclonal antibodies. The antibody gold conjugate binds to the antigens of the test band. In a test, a urine sample is applied, by dipping or a dropper, and migrates through the absorbent material, past the antibody gold conjugate portion and the bands. When the hCG concentration in the sample is less than 2 IU/ml the test band turns purple when the sample passes it. If the hCG concentration in the sample is higher than 2 IU/ml the antibody gold conjugate binds to hCG antigens in the urine sample and is inhibited and the test band will not show color. That band will not become purple. When the sample passes the control band, that band will become purple regardless of the level of hCG concentration in the sample because the antibody gold conjugate binds to the reagents coated on that band and the color change indicates that the reagents and test materials are functioning correctly. No color change in the control band indicates that there is not enough urine in the sample. The test must be interpreted between 5 and 10 minutes after the introduction of the urine sample. Results are indicated as follows:

If the hCG concentration in the sample is less than 2 IU/ml both the test and control bands become purple/red in less than 5 minutes, i.e. the test is negative.

Color change of only the control band and not the test band indicates that the test is positive.

No color change of control and test bands indicates that the test is invalid, due to any of a variety of causes.

The subject invention can also be defined as a method for testing for abortion safety based on detection of 2 IU/ml or more of gonadtropin in a urine sample having a concentration of 2 or more IU/ml of hCG antigens, the method comprising the steps of:

a) providing a portion of labeled antibody gold conjugate, said conjugate being such that it binds to said antigens in said urine in concentration greater than 2 IU/ml;

b) providing a first test band coated with hCG antigens;

c) providing a second band coated with goat/rabbit polyclonal antibodies;

d) providing means for exposing the urine sample, in sequence, to the labeled antibody gold conjugate, the first band and the second band.

In another embodiment of the method the second band is coated with polyclonal antibodies.

The invention is described in more detail below with reference to the attached drawings.

### BRIEF DESCRIPTION OF THE DRAWINGS

FIG. 1 illustrates a dipstrip, exploded for clarity.

FIG. 2 illustrates a cassette enclosing a dipstrip with a urine sample being applied to the sample well in the cassette with a dropper.

FIG. 3 illustrates the cassette indicating a positive result; i.e. the sample having hCG concentration greater than 2 IU/ml.

FIG. 4 illustrates the cassette indicating a negative result; i.e. the sample having hCG concentration less than 2 IU/ml.

FIG. 5 illustrates the cassette indicating an invalid test.

### DETAILED DESCRIPTION OF THE INVENTION

The subject invention is a test which enables rapid detection of 2 IU/mi or more of human chorionic gonadtropin (hCG) in a urine sample. The test is based on antibody function. The test kits for using the test each comprise a dipstrip and, in another embodiment, a cassette and a dropper. The dipstrip comprises a backing sub-strip, an absorbent materials sub-strip, a cover sub-strip having a clear portion termed a window and a portion of labeled antibody gold conjugate. The sub-strips are laminated with a short portion of the absorbent materials exposed at the dipping end of the dipstrip. The window may have a T and a C on its inner side indicating the locations of the test band and control band respectively.

In performing a test the dipping end of the dipstrip is dipped into a urine sample. The portion of labeled antibody gold conjugate is held between the absorbent materials sub-strip and the cover sub-strip and between the window and the dipping end. The dipstrip may be installed in a cassette having an opening which corresponds to the window of the installed dipstrip. There may be a T and a C on the outside of the cassette, possibly correlating with the T and C on the window. When the dipstrip is installed in a cassette a test is done by applying the sample, by a dropper, into a well opening which corresponds to the dipping end of the dipstrip. The test bands are not visible except under certain test conditions and are transverse to the strip.

The test band is coated with hCG antigens. The control band is coated with reagents such as goat/rabbit polyclonal antibodies. The antibody gold conjugate binds to the antigens of the test band. In a test, a urine sample is applied, by dipping or a dropper, and migrates through the absorbent material, past the antibody gold conjugate portion and the bands. When the hCG concentration in the sample is less than 2 IU/ml the test band turns purple when the sample passes it. If the hCG concentration in the sample is higher than 2 IU/ml the antibody gold conjugate binds to hCG antigens in the urine sample and is inhibited and the test band will not change color. That band will not become purple. When the sample passes the control band, that band will become purple regardless of the level of hCG concentration in the sample because the antibody gold conjugate binds to the reagents coated on that band and the color change indicates that the reagents and test materials are functioning correctly. No color change in the control band indicates that there is not enough urine in the sample. The test must be interpreted between 5 and 10 minutes after the introduction of the urine sample. Results are indicated as follows:

If the hCG concentration in the sample is less than 2 IU/ml both the test and control bands become purple/red in less than 5 minutes, i.e. the test is negative.

Color change of only the control band and not the test band indicates that the test is positive.

No color change of control and test bands indicates that the test is invalid, due to any of a variety of causes.

The subject invention can also be defined as a method for testing for abortion safety based on detection of 2 IU/ml or more of gonadtropin in a urine sample having a concentration of 2 IU/ml of hCG antigens, the method comprising the steps of:

a) providing a portion of labeled antibody gold conjugate, said conjugate being such that it binds to said antigens in said urine in concentration greater than 2 IU/ml;

b) providing a first test band coated with hCG antigens;

c) providing a second band coated with goat/rabbit polyclonal antibodies;

d) providing means for exposing the urine sample, in sequence, to the labeled antibody gold conjugate, the first band and the second band.

In another embodiment of the method the second band is coated with polyclonal antibodies.

The absorbent material comprises two components, both filter paper. Type I is cotton fiber with a wet strength of 70. Type II is glass fiber.

The window in the cassette comprises cellulose nitrate having a pore size of 5 um to 10 um and having a wicking rate of 90 sec/4 cm.

A labeled antibody gold conjugate is embedded in the dipstrip, is absorbed by specimens introduced and binds to the hCG antigens coated on the test band if the hCG concentration is 2 IU/mi or more. If the hCG is higher than 2 IU/ml the hCG antigens in the sample bind to the antibody gold conjugate instead of to the antigens coated on the test zone. The control band antibodies are polyclonal antibodies against mouse monoclonal antibodies.

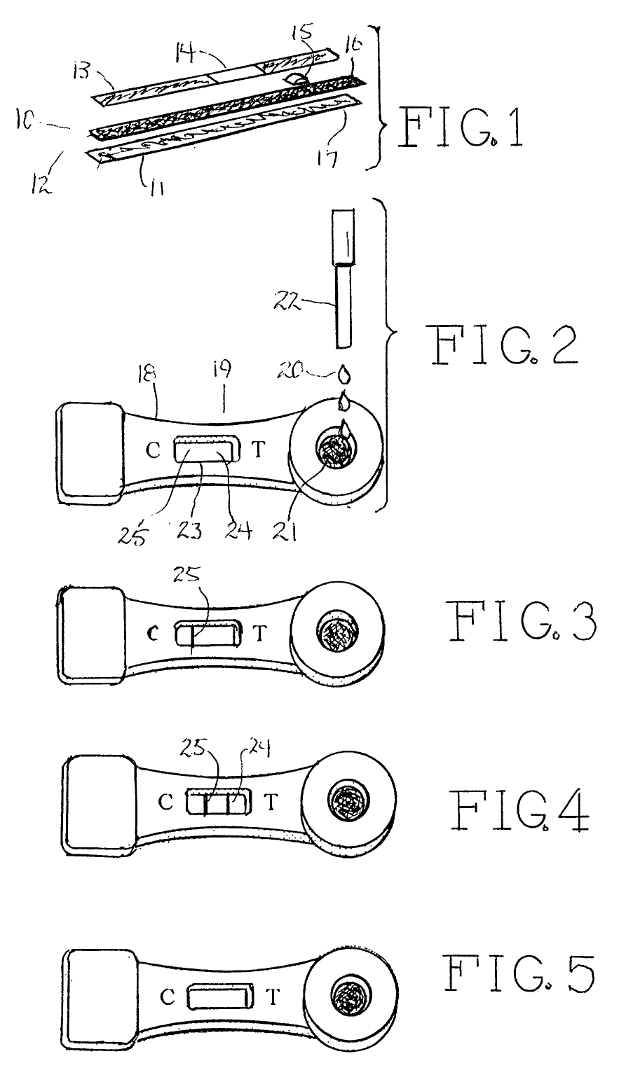

Referring to the attached drawings, FIG. 1 illustrates a dipstrip 10, exploded for clarity. The dipstrip comprises a base sub-strip 11, an absorbent materials sub-strip 12, a cover sub-strip 13 having a clear portion 14 and a portion of antibody gold conjugate 15. Portion 16 of the absorbent materials sub-strip is exposed; i.e. not covered by the cover sub-strip. The gold conjugate is between the cover sub-strip and the absorbent materials sub-strip and also between the clear portion of the cover sub-strip and exposed portion 16 of the absorbent materials sub-strip. End 17 of the dipstrip is termed its dipping end.

FIG. 2 illustrates a cassette 18 enclosing a dipstrip 19 (not visible) with a urine sample 20 being applied to the sample well 21 of the cassette with a dropper 22. Opening 23 in the cassette corresponds to the clear portion of the cover sub-strip of the dipstrip. Test band 24 and control band 25 will become visible under test conditions as described below. The letter T marked on the cassette indicates the location of test band and the letter C indicates the location of the control band.

FIG. 3 illustrates the cassette indicating a positive result, i.e. the sample has an hCG concentration greater than 2 IU/ml, abortion is safe.

FIG. 4 illustrates the cassette indicating a negative result, i.e. the sample has an hCG concentration less than 2 IU/ml, abortion is not safe.

FIG. 5 illustrates the cassette indicating that the test was invalid for any of a variety of reasons.

Regarding use of the subject invention, there are certain precautions. The test kit should be stored at room temperature. The test device is sensitive to humidity as well as to heat. Perform the test immediately after removing the test device from the foil pouch. Do not use it beyond the expiration date. It is for in vitro diagnostic use only. Dispose of all reaction devices in a proper biohazard container. Patient specimens may contain infectious agents and should be handled as potential pathogens.

The test kit may be stored at room temperature for up to 24 months or until the expiration date.

Regarding urine collection and storage, first in the morning urine typically contains the highest concentration of hCG and is therefore the best sample for performing the urine test. However, any urine specimen may be used at any time. The urine specimen must be collected in a clean glass or plastic container. Do not use preservatives. If the specimen is not to be used immediately following collection, but is to be used within 48 hours it should be refrigerated (2 to 8 degrees C), and brought back to room temperature (15 to 30 degrees C) before testing. If specimen is not going to be used within 48 hours, it should be frozen at −20 degrees C. A frozen specimen should not be used if stored longer than two weeks. Prior to testing, the frozen specimen must be completely thawed, thoroughly mixed, and brought to room temperature.

There are certain limitations in the use of the subject invention. It is used as an aid in diagnoses of a certain stage of pregnancy. Also, in addition to pregnancy, hCG has been found in patients with both gestation and non-gestation trophoblastic diseases. These conditions should be ruled out when interpreting hCG levels to establish a pregnancy diagnosis.

Although the test can detect hCG levels higher than of 2 IU/ml, a low incidence of false results can occur. A physician should be consulted if unexpected or inconsistent results are obtained.

A normal pregnancy cannot be distinguished from an ectopic pregnancy based solely on hCG levels. Also, a spontaneous miscarriage may cause confusion in interpreting test results.

As with all diagnostic tests, a definitive clinical diagnosis should not be based on the results of a single test, but should only be made by the physician after all clinical and laboratory findings have been evaluated.

A negative result obtained from a urine specimen collected from a mother in very early pregnancy may be due to a low concentration of hCG. In such cases, the test should be repeated on a fresh specimen obtained several days later.

If a urine sample is too dilute (i.e. low specific gravity), it may not contain a representative urinary hCG concentration. If a negative result is obtained with a low specific gravity specimen and pregnancy is still suspected, obtain a first morning urine specimen and retest.

Regarding performance characteristics of the invention, the sensitivity of the subject test is 2 IU/ml. This sensitivity level has been confirmed with interval hCG standards in urine, calibrated against the 2^{nd} International Standard. The following components explain the specificity of the invention. The ability of the dBest hCG Inibition Test to specifically detect hCG was challenged through cross-reaction studies on urine samples containing known quantities of structurally and physiologically related hormones. Urine samples spiked with 500 mIU/ml LH (human Luteinizing Hormone), 1000 mIU/ml FSH (Follicle Stimulating Hormone), 1000 μIU/ml TSH (Thyroid Stimulating Hormone) show negative results only.

Regarding interference of drugs, protein and glucose, potentially interfering drugs, protein and glucose were supplemented to normal urine specimens devoid of hCG and baseline urine level, as well as 2 IU/ml hCG standards, were then analyzed and compared with all samples containing a specific concentration of an interfering substance, including:

Acetaminophen, 20 mg/dl

Acetylsalicylic acid, 20 mg/dl

Ascorbic acid, 20 mg/dl

Atropine, 20 mg/dl

Caffeine, 20 mg/dl

Gentistic acid, 20 mg/dl

Glucose, 2000 mg/dl

Hemoglobin, 500 mg/dl

Mestranol, 3 mg/dl

Penicillin, 40,000 U/dI

Protein, 2000 mg/dl

It was concluded that all of the above substances have no interference with the use of the subject invention.

Regarding accuracy of the invention, it was compared with commercially available urine tests as tabulated:

| Patients' urine | AmeriTek hCG Test Kit | Commercial hCG Test Kit |
|---|---|---|
| Positive 51 | 51 | 45 |
| Negative 39 | 39 | 45 |
| Total 90 | 90 | 90 |

It is considered to be understandable from this disclosure that the subject invention meets its objectives. It provides a rapid test for determining an optimal time period during which a woman may have an abortion.

It is also considered to be understood that while certain embodiments of the subject invention are disclosed herein, other embodiments and modifications of those disclosed are possible within the scope of the invention which is limited only by the attached claims.

I claim:

1. A method for testing for abortion safety based on detection of 2 IU/ml or more of gonnnadtropin in urine samples having a concentration of hCG antigens in a range of 2 IU/ml or more, said method comprising the steps of:
a) providing a portion of labeled antibody gold conjugate, said conjugate being such that it binds to said hCG antigens in urine concentrations greater than 2 IU/ml;
b) providing a first band coated with hCG antigens;
c) providing a second band coated with goat/rabbit polyclonal antibodies;
d) providing means for exposing said urine sample, in sequence, to said labeled antibody gold conjugate, said first band and said second band.

2. The method of claim 1 in which step c is: providing a second band coated with mouse monoclonal antibodies.

3. A test kit comprising a dipstrip for use in testing for abortion safety based on detection of 2 IU/mi or more of gonadtropin in a urine sample, said sample having a concentration of 2 or more IU/ml of hCG antigens, said dipstrip having a dipping end and comprising:
a base sub-strip;
an absorbent materials sub-strip;
a cover sub-strip having a clear portion and
a portion of labeled antibody gold conjugate;
said clear portion having a first transverse band coated with hCG antibodies and a second transverse band coated with goat/rabbit polyclonal antibodies;
said portion of labeled antibody gold conjugate being installed between said absorbent materials sub-strip and said cover sub-strip and between said clear portion and said dipping end, said conjugate being such that it binds to said hCG antigens in said urine in concentrations greater than 2 IU/ml, a portion of said absorbent materials sub-strip being exposed at said dipping end,
whereby, in use of said dipstrip, said dipping end is dipped in said urine sample and said urine migrates through said absorbent materials sub-strip past, in sequence, said portion of labeled antibody gold conjugate, said first transverse band and said second transverse band.

4. The test kit of claim 3 in which said second transverse band is coated with mouse monoclonal antibodies.

5. The test kit of claim 3 further comprising a dropper and a cassette having a first opening corresponding to said clear portion and a second opening corresponding to said dipping end, said dipstrip being installed in said cassette with said clear portion corresponding to said first opening and said dipping end corresponding to said second opening, said sample being applied to said dipping end by said dropper through said second opening.

6. The test kit of claim 4 further comprising a dropper and a cassette having a first opening corresponding to said clear portion and a second opening corresponding to said dipping end, said dipstrip being installed in said cassette with said clear portion corresponding to said first opening and said dipping end corresponding to said second opening, said sample being applied to said dipping end by said dropped through said second opening.
